(19) United States
(12) Patent Application Publication
(10) Pub. No.: US 2010/0076277 A1
(43) Pub. Date: Mar. 25, 2010

(54) WIRELESS BIO-SIGNAL MONITORING SYSTEM

(76) Inventors: Chii-Wann LIN, Taipei City (TW); Chien-Sheng Liu, Taichung City (TW); Chin-Liang Liu, Taipei City (TW); Ching-Hua He, Taipei County (TW)

Correspondence Address: Muncy, Geissler, Olds & Lowe, PLLC, P.O. BOX 1364, FAIRFAX, VA, 22038-1364, US

(21) Appl. No.: 12/350,421

(22) Filed: Jan. 8, 2009

(30) Foreign Application Priority Data
Sep. 23, 2008 (TW) 97136554

(51) Int. Cl. A61B 5/00

(52) U.S. Cl. 600/301

(57) ABSTRACT

The present invention discloses a wireless bio-signal monitoring system for monitoring a patient's bio-signal. The system includes a bio-signal capture unit, an analysis unit, a wireless transmission unit, a receiver unit and a remote database. With a chaotic phase space difference algorithm, the consumption of system resources can be minimized and the power management of the monitoring system can be optimized. By capturing position coordinates of the receiver unit via the Internet, we can locate the position of the patient to provide medical treatments immediately in an emergency situation.

### FIELD OF THE INVENTION

The present invention relates to a bio-signal monitoring system, and more particularly to a wireless bio-signal monitoring system.

### BACKGROUND OF THE INVENTION

As the older population segment increases and the function of human organs deteriorates with age, it is very important for older people to monitor their disease resistance and physiological functions. Particularly for patients of chronic illnesses, a long-term tracing or monitoring of the patient's condition is required, so that appropriate first aids and treatments can be provided to a patient more quickly during emergency. Obviously, the patient's conditions must be controlled and maintained all the time.

In general, traditional biological monitoring equipments transmit a monitoring data through cables, and the distance between a transmitter end and a receiver end is limited by the length of the cables, and the scope of applications of these biological monitoring equipments is restricted.

In present biological monitoring equipments, the concept of wireless transmissions is introduced to expand the scope of applications of the biological monitoring equipments to a great extent, and related prior arts including R.O.C. Patent No. I290036 entitled “Bio-signal monitoring apparatus” were disclosed. The foregoing issued patent converts the kinetic energy of vibrations of a human body into electromagnetic energy to be used as a power source of a wireless transmission module. However, there is a considerable amount of power loss, since the monitoring device uses up the system resource substantially during the process of analyzing bio-signals. In emergency situations, data cannot be transmitted efficiently or normally due to insufficient power, and severe consequences may result.

In another prior art, “Portable biological monitoring apparatus and system” as disclosed in R.O.C. Pat. No. M298980 use a global positioning system (GPS) receiving module to receive a GPS signal in order to locate a patient's position, but the monitoring apparatus of this prior art goes through the processes of receiving satellite signals and computing locations, and thus consumes a great deal of power, and it is difficult to maintain an efficient power management of the monitoring apparatus.

Obviously, we need a high-performance biological monitoring system for monitoring a patient's condition reliably and precisely.

### SUMMARY OF THE INVENTION

Therefore, it is a primary objective of the present invention to provide a wireless bio-signal monitoring system capable of lowering the power consumption in the process of analyzing bio-signals, optimizing the power management of a monitoring device, and using the position of an access point on a network to locate the position of a patient.

To achieve the foregoing objective, the present invention provides a wireless bio-signal monitoring system comprising: a bio-signal capture unit, for capturing at least one bio-signal of a patient; an analysis unit, coupled to the bio-signal capture unit, for controlling the cycle of transmitting the bio-signal, analyzing the bio-signal by a chaotic phase space difference algorithm, and immediately sending out the bio-signal during emergency; a wireless transmission unit, coupled to the analysis unit through a standard transmission interface, and linked to a first network layer automatically; a receiver unit, being an access point on the first network layer, for receiving the bio-signal and transmitting the bio-signal to a remote database through a second network layer, wherein the remote database can record the bio-signal for capturing position coordinates of the receiver unit in order to locate the position of the patient, and the bio-signal capture unit, the analysis unit and the wireless transmission unit constitutes a monitoring device.

In the invention, the wireless transmission technology is adopted to expand the scope of applications of the monitoring system, and the chaotic phase space difference algorithm can lower the consumption of system resources and optimize the power management of the monitoring device. The invention further makes use of the function of capturing the address of the access point to locate the position of the patient by performing a position computation in the remote database, so as to notify medical professionals for a timely treatment, reduce the high consumption of power of the traditional GPS receiving module, and achieve a reliable and efficient monitoring effect.

### BRIEF DESCRIPTION OF THE DRAWING

FIG. 1 is a functional block diagram of a wireless bio-signal monitoring system in accordance with the present invention.

### DESCRIPTION OF THE PREFERRED EMBODIMENT

To make it easier for our examiner to understand the purposes, characteristics and effects of the present invention, preferred embodiments together with related drawings are used for the detailed description of the invention as follows:

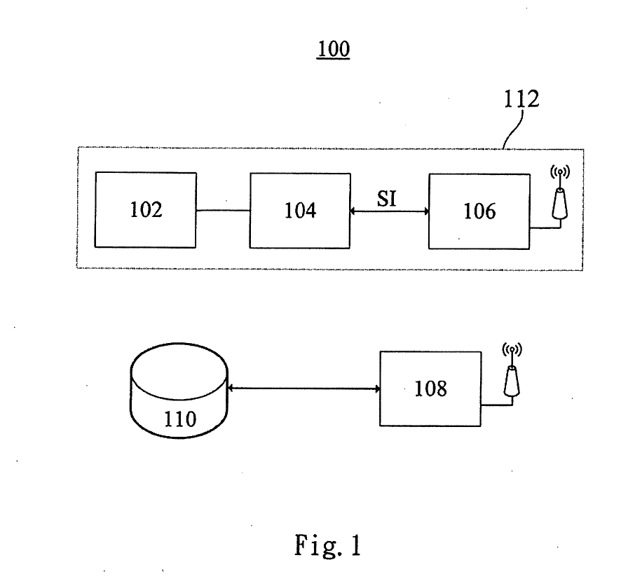

With reference to FIG. 1 for a functional block diagram of a wireless bio-signal monitoring system in accordance with the present invention, the wireless transmission bio-signal monitoring system 100 comprises a bio-signal capture unit 102, an analysis unit 104, a wireless transmission unit 106, a receiver unit 108 and a remote database 110, wherein the bio-signal capture unit 102, the analysis unit 104 and the wireless transmission unit 106 constitute a monitoring device 112. The bio-signal capture unit 102 comprises a bio-sensor (not shown in the figure), and the bio-sensor can be a body temperature sensor, a tri-axis accelerometer, a blood oxygen concentration sensor, a blood pressure sensor or a breath frequency sensor. The bio-signal capture unit 102 may have a plurality of sensors for capturing various different bio-signals of a patient. The bio-sensor captures a patient's biological parameters in the form of analog bio-signals, and the analog bio-signals are processed by the bio-signal capture unit 102 and converted into digital bio-signals. The digital bio-signals are recorded in a memory device of the bio-signal capture unit 102 and transmitted to the analysis unit 104 for the analysis of biological parameters.

A time interval can be set for the analysis unit 104, such that the analysis unit 104 will send out the bio-signal according to the set time interval. In addition to the transmission of signals at a fixed time interval, the analysis unit 104 also can send out the bio-signal at the time computed by a chaotic phase space difference (CPSD) algorithm, whenever an abnormal condition occurs. The analysis algorithm comprises the steps of:

(a) setting a numeric range of related parameters according to characteristics of different bio-signals, and selecting an appropriate setting of parameters to create a reference matrix;

(b) creating an analysis matrix according to the bio-signal captured by the bio-signal capture unit 102;

(c) subtracting a value of the reference matrix from a value of the analysis matrix to obtain a value of a resultant matrix;

(d) computing a numeric value of the resultant matrix as a data number of the positive value, wherein the data number is a chaotic phase space difference (CPSD) value, and the mean and the standard deviation of the CPSD value are used for adjusting a threshold and its range automatically and appropriately, and the range is calculated by adding or subtracting three standard deviations from the mean; and

(e) determining whether or not the bio-signal is abnormal by checking whether or not the CPSD value exceeds the threshold and its range.

Each set parameter as described in Step (a) includes a data length, a time interval, a sampling rate, a phase space matrix size and a delay time. The analysis method uses a Time-Delay method to rebuild the phase space matrix and calculate the chaotic phase space difference of the phase space matrix as a basis for determining whether or not the bio-signal is normal.

Therefore, the analysis method not only provides a quick way for the analysis unit 104 to analyze the bio-signal, but also consumes less system resources. With the analysis algorithm, the fixed time interval for transmitting the bio-signal can be adjusted. In other words, the period can be changed by the chaotic phase space difference algorithm to optimize the power management of the monitoring device 112, and thus the invention can overcome the shortcomings of the prior art consuming more system resources, taking a longer time for the analysis that results in a higher power consumption. In addition, the optimized power management can extend the use of power supply, maintain monitoring a patient's condition with interruption, and provide a more reliable wireless transmission bio-signal monitoring system.

If the analysis unit 104 decides transmitting the bio-signal, the wireless transmission unit 106 will be connected to the analysis unit 104 through a standard transmission interface SI for receiving the bio-signal and then sending out the bio-signal through a wireless transmission interface (not shown in the figure). The standard transmission interface can be a universal asynchronous receiver/transmitter (UART), serial peripheral interface (SPI), I²C interface (I-squared-C) or RS232 interface. The wireless transmission interface can be a Bluetooth, Zigbee, wireless fidelity (Wi-Fi) or worldwide interoperability for microwave access (WiMAX) interface.

Before sending out the bio-signal, the wireless transmission interface is connected to a first network layer automatically, wherein the first network layer can be a local area network (LAN), an Intranet or an Extranet. The receiver unit 108 connected to the first network layer is an access point (AP) on the first network layer, and the receiver unit 108 is a receiving device such as a network server, a personal computer (PC) or a personal digital assistant (PDA) with a wireless network transmitting/receiving function for displaying, storing, transmitting and receiving bio-signals. Position coordinates including longitude and latitude are installed into the receiver unit 108, wherein the position coordinates indicate the position of the receiver unit 108, and the position coordinates can be located by a global positioning system (GPS).

The receiver unit 108 transmits the bio-signal and its position coordinate information to the remote database 110 through a second network layer, wherein the second network layer is the Internet, and the receiver unit 108 is connected to the Internet through a cable or a wireless transmission.

The remote database 110 is situated at a medical unit for recording the bio-signals. If an emergency situation occurs (indicated by an abnormal bio-signal), medical professionals will be notified to take proper actions and provide necessary assistance. In addition, the remote database 110 will capture the position coordinate information of the receiver unit 108. Due to the distance of the wireless transmission is limited, the precise location of the patient will not be far from the receiver unit 108, so that the position of the patient can be located quickly in an emergency situation.

In summation of the description above, the wireless bio-signal monitoring system of the invention provides a lower power consumption to optimize the power management of the bio-signal monitoring device and locates the position of the patient more quickly by capturing the address of the access point, so that the patient can be located easily in an emergency situation. Since the positioning and computation of the patient's position are carried out in the remote database, the invention avoids a high power consumption of the conventional monitoring device using a GPS receiving module for the positioning and computation, and achieves a reliable and high performance monitoring effect.

What is claimed is:

1. A wireless bio-signal monitoring system, comprising:
a bio-signal capture unit, for capturing at least one bio-signal of a patient;
an analysis unit, coupled to the bio-signal capture unit, for controlling a cycle of transmitting the bio-signal, analyzing the bio-signal by a chaotic phase space difference algorithm, and transmitting the bio-signal again when an abnormal condition occurs;
a wireless transmission unit, coupled to the analysis unit through a standard transmission interface, and capable of linking to a first network layer automatically; and
a receiver unit, being an access point on the first network layer, for receiving the bio-signal, transmitting the bio-signal to a remote database through a second network layer, and recording the bio-signal in the remote database.

2. The bio-signal monitoring system of claim 1, wherein the bio-signal capture unit, the analysis unit and the wireless transmission unit constitute a monitoring device.

3. The bio-signal monitoring system of claim 1, wherein the remote database captures position coordinates of the receiver unit for locating the position of the patient.

4. The bio-signal monitoring system of claim 1, wherein the bio-signal capture unit includes at least one bio-sensor and digitizes the captured bio-signal.

5. The bio-signal monitoring system of claim 4, wherein the bio-sensor is a body temperature sensor, a tri-axis accelerometer, a blood oxygen concentration sensor, a blood pressure sensor or a breath frequency sensor.

6. The bio-signal monitoring system of claim 1, wherein the wireless transmission unit further includes a wireless transmission interface for transmitting the bio-signal, and the wireless transmission interface is a Bluetooth, Zigbee, wireless fidelity (Wi-Fi) or worldwide interoperability for microwave access (WiMAX) interface.

7. The bio-signal monitoring system of claim 1, wherein the first network layer is a local area network (LAN), an Intranet or an Extranet.

8. The bio-signal monitoring system of claim 1, wherein the receiver unit is a network server, a personal computer (PC) or a personal digital assistant (PDA).

9. The bio-signal monitoring system of claim 1, wherein the second network layer is the Internet.

10. The bio-signal monitoring system of claim 1, wherein the remote database is situated at a medical unit for immediately notifying a medical professional for medical treatments if the bio-signal is an abnormal signal.
